(19) United States
(12) Patent Application Publication
(10) Pub. No.: US 2009/0182135 A1
(43) Pub. Date: Jul. 16, 2009

(54) Amino modifiers

(76) Inventors: Mikhail S. Shchepinov, Oxford (GB); Pablo Bernad, Oxford (GB)

Correspondence Address: WENDEROTH, LIND & PONACK, L.L.P., 1030 15th Street, N.W.,, Suite 400 East, Washington, DC, 20005-1503, US

(21) Appl. No.: 12/149,357

(22) Filed: Apr. 30, 2008

Related U.S. Application Data
(60) Provisional application No. 60/924,094, filed on Apr. 30, 2007.

(51) Int. Cl. C07H 21/00; C07C 43/23; C07C 211/27; C07F 9/06; C07H 1/00

(52) U.S. Cl. 536/23.1; 568/640; 564/321; 564/12; 536/25.3

(57) ABSTRACT

The present invention provides a compound of formula (1):

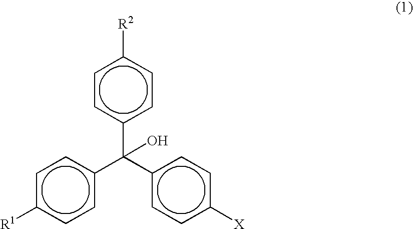

wherein: X is an electron-donating group, amino modifiers formed by reacting an amino alcohol with compounds of formula (1), amino-modified biomolecules formed by reacting said amino modifiers with a biomolecule and methods for producing the same.

### TECHNICAL FIELD

The present invention relates to compounds useful in the functionalisation of biomolecules, in particular polynucleotides and methods for synthesizing the same.

### BACKGROUND OF THE INVENTION

The primary recognition event of polynucleotide sequence-based detection techniques is the non-covalent binding of a probe to a complementary sequence of a target (hybridisation), brought about by a precise molecular alignment and interaction of complementary nucleotides. To detect this hybridisation it is usual either to modify the target or probe to allow immobilisation onto a solid support, to incorporate a signalling moiety group, or both events.

There are a wide variety of bridging moieties that may be employed for covalent attachment of polynucleotides to signalling labels or to solid supports.

An efficient and well established technique involves the functionalisation of the 5′-terminus of a single stranded polynucleotide by a chemical reaction, using a suitably protected chemical moiety which can readily be coupled at the 5′-terminus of protected polynucleotide during phosphoramidite synthesis. This technique offers considerable advantages over other possible options as such a location causes little or no destabilisation of the hybridised polynucleotide as compared to the unlabelled form. Additionally, the 3′-end is free for use as a primer in enzyme-mediated extension reactions.

Amino-modified polynucleotides have been routinely employed in solid support and label attachment chemistries, in particular in functionalisation of the 5′-terminus of a polunucleotide. An amino modifier reagent comprises a primary amine which is protected with a protecting group and has at its other end a functionality that will react with a polynucleotide. The protecting group is necessary in order to ensure that, upon reaction with the nucleotide, the correct reaction takes place.

Polynucleotides functionalised with an amino group have found multiple applications in both basic and applied molecular biology, such as for diagnostic procedures, automated sequencing, electron microscopy, fluorescence microscopy, x-ray crystallography, hybridization affinity chromatography and probing of nucleic acid structure. They are predominantly used in the manufacture of microarrays. The process of microarray manufacturing by post-synthetic immobilization has been optimized mainly in terms of immobilization chemistries, with the most popular methods relying on the reaction of amino group-terminated polynucleotides with surfaces derivatized with moieties such as isothiocyanate, NHS-activated carboxy, or epoxy groups

The most popular amino-modifier used today contains an amine protected by a trifluoroacetic acid (TFA) group. This modifier is inexpensive and reliable. However, it has several drawbacks and in particular, since the removal of a TFA protecting group happens simultaneously with the deprotection of an polynucleotide, side reactions such as Michael addition of acrylonitrile (formed from elimination of cyanoethyl protecting groups) take place, reducing the yield of the aminated product.

Triphenylmethyl groups (trityls) are an alternative family of protecting groups, used in polynucleotide chemistry for a hydroxyl and amino protection. They are removable by mild acidic treatment. Conveniently, trityl cations have large extinction coefficients allowing stepwise coupling yields to be measured easily.

Two examples of commercially available trityl protecting groups are the monomethoxytrityl (MMTr) group and the 4,4′-dimethoxytrityl (DMTr) group. There are several significant disadvantages associated with the use of such protecting groups.

First, cheap and fast purification techiniques are required in the manufacture of a large number of polynucleotides. As a consequence, the reverse phase (RP) cartridge purification technique is preferred. RP cartridges comprise a hydrophobic matrix and separate compounds on the basis of hydrophilicity and lipophilicity. However, it has been found that where this purification technique is used, it is not possible to remove the MMTr or DMTr protecting group from the polynucleotide. On a reverse phase cartridge, the trityl cation is not physically separated from the amine, and as the acidic cleavage for the trityl-N bond is an equilibrium reaction, there is a tendency for the protecting group to reattach to the amine. This can result in a product which is up to 50% inactive. This is particularly a problem where the protecting group is MMTr.

Second, it has been found that MMTr amino-modified polynucleotides are prone to degrade over time under normal storage and manipulation conditions due to a low stability.

Finally, complete removal of these trityl groups requires relatively strong acidic conditions. The use of strongly acidic conditions is undesirable as it increases the likelihood that unwanted side-reactions will occur.

There is hence a need for new amino modifiers, which can be used to produce amino-modified polynucleotides in high yield while allowing the use of cheap and fast purification techniques. Further there is a need for amino modifiers which comprise a protecting group which can be easily removed from the amino group yet are sufficiently stable to allow storage over a prolonged period of time.

### DISCLOSURE OF THE INVENTION

In this regard, the present invention provides novel trityl alcohols which can be used in the synthesis of new amino modifier reagents for the synthesis of amino-modified biomolecules.

Advantageously, the present inventors have found that where the compound has a pK_{R+} in the range from −3.1 to −1.5, the trityl cation has a stability which is such that when used as a protecting group, it can be easily removed from an amino functionality. Additionally, the trityl group is sufficiently stable that it can be used as a protecting group for an amino functionality and can be stored for extended periods of time with no or little degradation in stability.

More specifically, the present invention provides a compound of formula (1):

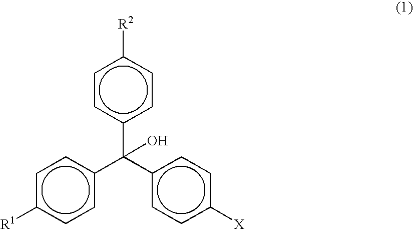

Wherein: X is an electron-donating group;

R¹ and R² are each independently selected from hydrogen, halogen, C₁₋₁₀ hydrocarbyl, C₁₋₁₀ hydrocarbyl substituted with one or more A¹, C₂₋₁₀ hydrocarbylene, C₁₋₁₀ hydrocarbylene substitutued with one or more A¹, trihalomethyl, —NO₂, —CN, —N⁺(R³)₂O⁻, —CO₂H, —CO₂R³, —SO₃H, —SOR³, —SO₂R³, —SO₃R³, —OC(═O)OR³, —C(═O)H, —C(═O)R³, —OC(═O)R³, —NR³₂, —C(═O)NH₂, —C(═O)NR³₂, —N(R³)C(═O)OR³, —N(R³)C(═O)NR³₂, —OC(═O)NR³₂, —N(R³)C(═O)R³, —C(═S)NR³₂, —NR³C(═S)R³, —SO₂NR³₂, —NR³SO₂R³, —N(R³)C(═S)NR³₂, —N(R³)SO₂NR³₂, —R³ or -Z¹R³;

Z¹ is O, S, Se or NR³;

R³ is independently H, C₁₋₁₀hydrocarbyl, C₁₋₁₀hydrocarbyl substituted with one or more A¹, C₁₋₁₀heterohydrocarbyl; C₁₋₁₀heterohydrocarbyl substituted with one or more A¹; C₂₋₁₀ hydrocarbylene; or C₂₋₁₀hydrocarbylene substituted with one or more A¹;

A¹ is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z¹C₁₋₆alkyl and

the compound has a pK_{R+} in the range from −3.10 to −1.50.

In a second aspect the present invention provides a method of producing a compound of formula (1) comprising reacting a compound of formula (4):

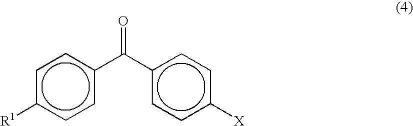

with a Grignard reagent of formula (5):

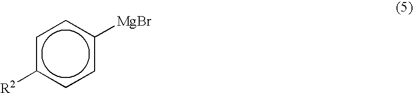

wherein X, R¹ and R² are as defined above.

In a third aspect, the present invention provides an amino modifier reagent of formula (2):

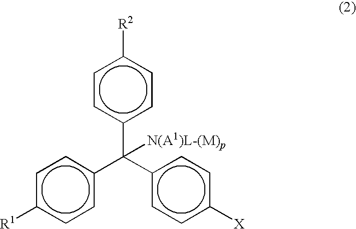

Wherein: X, R¹, R² and A¹ are as defined above;

L is a linker group;

M is a reactive functional group; and

p is an integer having a value in the range from 1 to 10.

In a fourth aspect, the present invention provides a method for producing compounds of formula (2) comprising

(a) reacting a compound of formula (1) with acetyl chloride;

(b) reacting the product of step (a) with an amino alcohol, wherein the hydroxyl group of the amino alcohol has been protected; and

(c) removing the protecting group from the hydroxyl group of the product of step (b) to produce a compound of formula (2).

In a fifth aspect, the present invention provides an amino-modified biomolecule of formula (3).

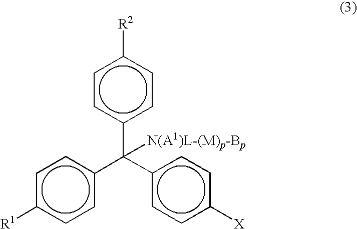

wherein X, R¹, R², A¹, L, M and pare as defined above; and Bₚ is a biomolecule.

In a sixth aspect, the present invention provides a method of producing an amino-modified biomolecule comprising reacting a compound of formula (2) with a biomolecule, Bₚ having at least one group capable of reacting with M to form a covalent linkage.

In a seventh aspect, the present invention provides a method of deprotecting a amino-modified biomolecule of formula (3).

pK_{R+}

The physico-chemical properties of trityl cations have been thoroughly investigated. In 1955 Deno et al reported an empirical Acidity Function HR which allows the comparison of the stability of different trityl cations (N. C. Deno, J. J. Jaruzelski and A. Schriesheim, J. Am. Chem. Soc. 1955, 77, 3044). Using the acidity function and the methods described by Deno it is possible to evaluate the pK_{R+} parameter for trityl alcohol derivatives, which is a direct indication of the stability of the corresponding trityl cations.

H_{R}=pK_{R+}−log([R+]/[ROH])

The acidity function HR for a collection of aqueous sulfuric acid solutions was tabulated. The acidity function HR is an intrinsic property of the aqueous sulfuric acid solutions and provides a reference for its “strength” to ionize trityl alcohols.

Once the acidity function HR for a particular acidic solution is known, the pK_{R+} of a triarylcarbinol dissolved in that acidic solution can be calculated in a straightforward manner once the ratio [R⁺]/[ROH] has been experimentally determined. Therefore, in order to determine pK_{R+} values in acidic solutions of known H_{R}, the ratio [R⁺]/[ROH] must be established or, what is the equivalent, the degree of ionization of the triarylcarbinol.

The [R⁺]/[ROH] ratio can be determined by spectrophotometic means. In particular, for trityl derivatives, UV spectrophotometry is useful since trityl cations and their parent molecules show completely different absorption spectra in the UV region.

The UV spectra of compounds are obtained by passing light of a given wavelength through a dilute solution of the substance in a non-absorbing solvent.

The intensity of the absorption is measured by the percent of the incident light that passes through the sample

% Transmittance=(I/I₀)×100

where

I=intensity of transmitted light

I₀=intensity of incident light

As light absorption is a function of the concentration of the absorbing molecules, a more precise way of reporting intensity of absorption is by use of the Beer-Lambert Law:

Abs=−log(I/I₀)=εcl

where

Abs=absorbance=−logT

ε=molar absorptivity

l=length of sample cell (cm)

In the particular case of a triaryl alcohol partially ionized in an acidic solution, the solute exists in two forms, ROH and R⁺ according to the equilibrium

R⁺+H₂O [symbol] ROH+H⁺

The total concentration of trityl species is equal to the sum of the concentration of these two forms

[ROH]₀=[ROH]+[R⁺]

It is the case that each substance absorbs independently of the presence of each other, and therefore:

Abs=ε[ROH]₀l=(εp_{R}[R]+ε_{ROH}[ROH])l

and

ε([ROH]+[R⁺])=(ε_{R}[R]+ε_{ROH}[ROH])

### Thus,

$$\frac{[ R ]}{[ ROH ]} = \frac{ɛ - {ɛ}_{ROH}}{{ɛ}_{R} - ɛ}$$

The values ε are measured in a particular wavelength. If it is the case that on the wavelength the trityl alcohol does not absorb (and this is the general case for the λₘₐₓ of triaryl cation species), then ε_{ROH}=0, so the final expression is given by

$$\frac{[ R ]}{[ ROH ]} = \frac{ɛ}{{ɛ}_{R} - ɛ}$$

In general, the experimental procedure for determining pK_{R+} for a trityl alcohol is summarised as follows. A solution of the triaryl alcohol in question in acetic acid is prepared in a way that the final concentration of the solutions to be analysed by UV spectrometry are in the range of 10⁻⁵ to 10⁻⁷ molar. A constant amount of the acetic acid solution so prepared is then added to the solutions of sulphuric acid (for instance, using a micropipette) to carry out the UV spectrometry experiments. It is recommended that the values of E are measured at the λₘₐₓ of the trityl cation.

Starting from the most concentrated sulphuric acid solutions, samples are prepared until it is observed that the absorbance of the samples starts to decay. In the range of concentrations prior to the decay of absorbance, the triaryl alcohol is dissolved in a sufficiently concentrated sulphuric to completely ionise it. Therefore, ε=ε_{R}. In the region of sulphuric acid concentrations in which the absorbance of the samples decays, a series of values for ε are collected.

Finally, a concentration of sulphuric acid is reached in which the absorbance at λₘₐₓ is negligible (or the alcohol starts to precipitate). This indicates the end of the experiment and values for the [R⁺]/[ROH] for a series of given H_{R} are available.

The pK_{R+} can be determined using the equation

$${pK}_{{R}^{+}} = {H}_{R} + log \frac{ɛ}{{ɛ}_{R} - ɛ}$$

derived from

$${H}_{R} = {pK}_{{R}^{+}} - log \frac{[ {R}^{+} ]}{[ ROH ]} = {pK}_{{R}^{+}} - log \frac{ɛ}{{ɛ}_{R} - ɛ}$$

The commercially available trityl protecting groups, MMTr and DMTr have pK_{R+} values of −3.40 and −1.24 respectively.

In contrast, the compound of formula (1) of the present invention has a pK_{R+} in the range from −3.10 to −1.50. For example, the compound of formula (1) has a pK_{R+} of at least about −a, where a is 2.8, 2.6, 2.5, 2.4, 2.2, 2.0, 1.8 or 1.6. For example, the compound of formula (1) has a pK_{R+} of less than about −b, where b is 1.6, 1.8, 2.0, 2.2, 2.4, 2.6, 2.8 or 3.0.Preferably the compound of formula (1) has a pK_{R+} in the range from −a to −b, for example, in the range from −2.8 to −1.6, from −2.6 to −1.8, from −2.5 to −2.0, from −2.4 to −2.2.

By having a pK_{R+} values in the range defined above, the compound has the appropriate balance between stability in a cation form and ease of removal when used as a protecting group for a primary alcohol.

In the compound of formula (1), one of the three aryl groups is para-substituted with an electron-donating group X. An electron-donating group is generally recognised by a lone pair of electrons an atom through which bonding to another a group will take place.

Preferably, the electron-donating group X is selected from the group consisting of —OR⁴, —NR⁴₂, —O—C(O)R⁴, —NHC(O)R⁴ wherein R⁴ is independently H, C₁₋₈hydrocarbyl, C₁₋₈hydrocarbyl substituted with one or more A¹, C₁₋₈heterohydrocarbyl or C₁₋₈heterohydrocarbyl substituted with one or more A¹ wherein A¹ is as defined above.

The substituents R¹ and R² on the compound of formula (1) are as defined above and may either be the same or different.

In a preferred embodiment, R¹ and R² are the same. Preferably where R¹ and R² are the same, X is -Z¹C₁₋₆alkyl and R¹ and R² are both C₁₋₁₀hydrocarbyl, preferably methyl. Thus the compound has the formula:

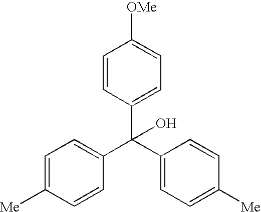

This compound has a pK_{R+} value of −2.6 and is therefore particularly suitable for use in the synthesis of an amino modifier according to the present invention.

In an alternative preferred embodiment, R¹ and R² are different. Preferably X and R¹ are -Z¹C₁₋₆alkyl, preferably —OCH₃ and R² is —SOR³, preferably, —SOCH₃. Thus the compound has the formula:

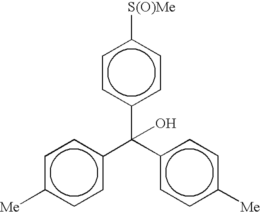

This compound has a pK_{R+} value of −2.5 and is therefore particularly suitable for use in the synthesis of an amino modifier according to the present invention.

Advantageously, the compounds of formula (1) are synthesized by reacting a compound of formula (4) with a Grignard reagent of formula (5).

Amino Modifiers

Advantageously, the compound of formula (1) of the present invention can be used to produce an amino modifier reagent which can, in turn be used in the synthesis of amino-modified biomolecules.

The amino modifiers of the present invention are prepared by reacting a compound of formula (1) with an amino alcohol to form a compound of formula (2).

The substituents R1, R2 and X on the trityl group of formula (2) are preferably selected such that the —OH substituted version of the trityl group would have a pK_{R+} value in the range from −3.1 to −1.5.

M

The group M is a reactive functional group. Reactive functional groups include groups capable of reacting to form a covalent linkage.

The group M is bound to L by one or more covalent bonds (e.g. 2 or 3 bonds, especially 2 such as

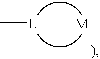

which are either single, double or triple covalent bonds (preferably single bonds).

Preferably, M is bound to L by one single bond.

Examples of group M bound to L by one bond include —NR₂ e.g. —NHR (e.g. —NHMe), especially —NH₂; —SR e.g. —SH; —OR e.g. —OH; —B(R)Y; —BY₂; —C(R)₂Y; —C(R)Y₂; —CY₃; —C(=Z)Y e.g. —C(═O)Y; -Z-C(=Z)Y; —C(=Z)R e.g —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(H)Y; -Z-P(=Z)(R)Y; or —N═C(=Z) e.g. —N═C(═O).

Another example of a group M bound to L by one bond is —CN.

Other examples of group M bound to L by one bond are —P(ZR)Y e.g. —P(OH)Y; —PY₂; -Z-P(ZR)Y; -Z-PY₂; —P(R)Y e.g. —P(H)Y; -Z-P(R)Y. A particularly preferred group M is -Z-P(ZR)Y, especially a phosphoramidite group:

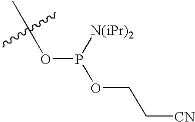

Examples of group M bound to L by two bonds include —N(R)— e.g —NH—; —S—; —O—; —B(Y)—; —C(R)(Y)—; —CY₂—; —C(═O)—; —C(OH)(OR)—; —C(OR)₂—.

Examples of group M bound to L by three bonds include

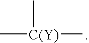

Preferred groups M include electrophilic groups, especially those susceptible to SN₂ substitution reactions, addition-elimination reactions and addition reactions, e.g. —B(R)Y; —BY₂; —C(R)₂Y; —C(R)Y₂; —CY₃; —C(=Z)Y e.g —C(═O)Y, especially —C(O)OH (e.g compound 24b) and —C(O)NH₂; -Z-C(=Z)Y; —C(=Z)R e.g. —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(R)Y; -Z-P(=Z)(H)Y; —N═C(=Z) e.g. —N═C(═O); —B(Y)—; —C(R)(Y)—; —CY₂—; —C(═O)—; —C(OH)(OR)—; —C(OR)₂—; or

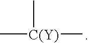

Another preferred electrophilic group M is —CN.

Still further preferred examples of group M are orthoesters, e.g. —C(OR)₃. In a preferred embodiment, the R groups are linked together to form a hydrocarbyl group, e.g. a C₁₋₈alkyl group. A preferred example of group M in this embodiment is:

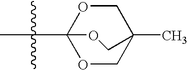

Another preferred group M is maleimido.

Y is independently a leaving group, including groups capable of leaving in an SN₂ substitution reaction or being eliminated in an addition-elimination reaction.

Examples of Y include halogen (preferably iodo), C₁₋₈hydrocarbyloxy (e.g. C₁₋₈alkoxy), C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl or a sulfo sodium salt thereof (sulfoNHS), —S-succinimidyl, or phenyloxy substituted with one or more A e.g. p-Nitrophenyloxy or pentafluorophenoxy.

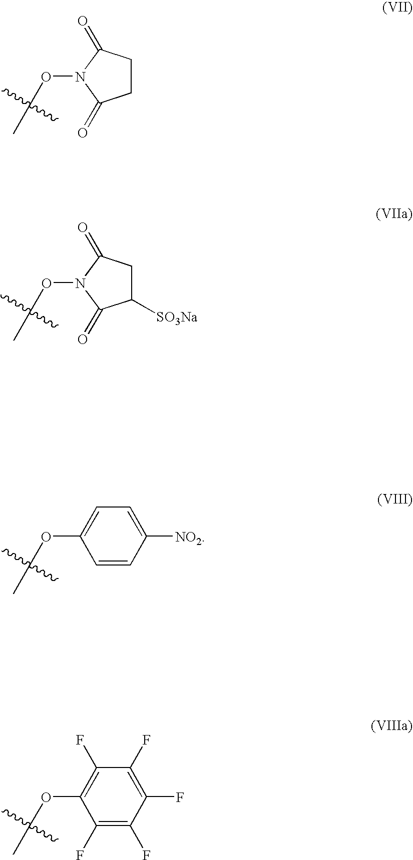

Other examples of Y include -ZR⁶. Particularly preferred examples of Y are -ZH (e.g. —OH or —NH₂) and -Z-C₁₋₈alkyl groups such as —NH—C₁₋₈alkyl groups (e.g. —NHMe) and —O—C₁₋₈alkyl groups (e.g. —O-t-butyl). Thus, preferred groups M are —C(O)—NH—C₁₋₈alkyl (e.g. —C(O)NHMe) and —C(O)—O—C₁₋₈alkyl (e.g —C(O)—O-t-butyl).

Other preferred examples of Y include -Z-ZR . Particularly preferred examples include —NR⁶—NR⁶₂, especially —NH—NH₂, and —ONR₂, especially —O—NH₂.

Z is independently O, S or N(R⁶). Preferred (=Z) is (═O).

R⁶ is independently H, C₁₋₈hydrocarbyl (e.g. C₁₋₈alkyl) or C₁₋₈hydrocarbyl substituted with one or more A.

R⁶ is preferably H.

Particularly preferred groups M include —C(═O)Y, especially —C(═O)—O-succinimidyl and —C(═O)—O-(p-nitrophenyl).

In a further embodiment, M may be —Si(R⁶)₂—Y, with Y being halo (e.g chloro) being especially preferred. Preferred groups R⁶ in this embodiment are C₁₋₈alkyl, especially methyl. A particularly preferred group M in this embodiments is —Si(Me)₂—Cl.

In a further embodiment, M may be —C(Ar²)₂X¹. Preferred groups Ar² and X¹ are set out below. In this embodiment it is preferred that L is a bond. A particularly preferred group M in this embodiment is:

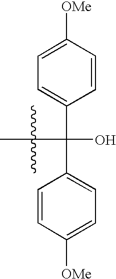

Other groups M include groups capable of reacting in a cycloaddition reaction, especially a Diels-Alder reaction.

In the case of Diels-Alder reactions, the group M is either a diene or a dienophile. Preferred diene groups are

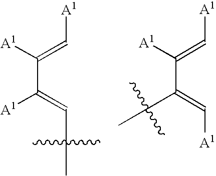

and multivalent derivatives formally formed by removal of one or more hydrogen atoms, where A¹ is —R³ or -Z¹R³, where R³ and Z¹ are defined above.

Preferred dienophile groups are —CR³═CR³₂, —CR³═C(R³)A², —CA²═CR³₂, —CA²═C(R³)A² or —CA²═CA²₂, and multivalent derivatives formally formed by removal of one or more hydrogen atoms, where R¹ is defined below and A² is independently halogen, trihalomethyl, —NO₂, —CN, —N⁺(R³)₂O—, —CO₂H, —CO₂R³, —SO₃H, —SOR³, —SO₂R³, —SO₃R³, —OC(═O)OR³, —C(═O)H, —C(═O)R³, —OC(═O)R³, —OC(═O)NR³₂, —N(R³)C(═O)R³, —C(═S)NR³₂, —NR³C(═S)R³, —SO₂NR³₂, —NR³SO₂R³, —N(R³)C(═S)NR³₂, or —N(R³)SO₂NR³₂, where R³ is defined above. A particularly preferred dienophile group is maleimidyl.

Ar²

Ar² is independently an aromatic group or an aromatic group substituted with one or more B¹ and is preferably independently cyclopropyl, cyclopropyl substituted with one or more A, aryl, aryl substituted with one or more A, heteroaryl, or heteroaryl substituted with one or more B¹.

Where aryl or substituted aryl, Ar² is preferably C₆₋₃₀ aryl or substituted C₆₋₃₀ aryl. Where heteroaryl or substituted heteroaryl, Ar² is preferably C₆₋₃₀ heteroaryl or substituted C₆₋₃₀ heteroaryl.

Examples of aryl and heteroaryl are monocyclic aromatic groups (e.g. phenyl or pyridyl), fused polycyclic aromatic groups (e.g. napthyl, such as 1-napthyl or 2-napthyl) and unfused polycyclic aromatic groups (e.g. monocyclic or fused polycyclic aromatic groups linked by a single bond, a double bond, or by a —(CH═CH)ᵣ— linking group, where r is one or more (e.g. 1, 2, 3, 4 or 5).

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene, which groups may be optionally substituted by one or more B¹.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene, which groups may be optionally substituted by one or more B¹. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene. The heteroaryl groups may be attached to the remainder of the compound by any carbon or hetero (e.g. nitrogen) atom.

Ar² is preferably C₆₋₃₀aryl substituted by one or more B¹, preferably phenyl or napthyl (e.g. 1-napthyl or 2-napthyl, especially 2-napthyl) substituted by one or more B¹, more preferably phenyl substituted by one or more B¹. Fused polycyclic aromatic groups, optionally substituted with one or more B¹, are particularly preferred.

A particularly preferred Ar² is unsubstituted pyrenyl or pyrenyl substituted with one or more B¹. Unsubstituted pyrenyl is preferred. The pyrenyl group may be 1-pyrenyl, 2-pyrenyl or 4-pyrenyl.

Preferred heteroaryl Ar² groups, whether substituted or unsubstituted, are pyridyl, pyrrolyl, thienyl and furyl, especially thienyl.

A preferred Ar² group is thiophenyl or thiophenyl substituted with one or more A. Unsubstituted thiophenyl is preferred. Examples of thiophenyl are thiophen-2-yl and thiophen-3-yl, with thiophen-2-yl being especially preferred. When substituted, Ar² is preferably substituted by 1, 2 or 3 B¹. Ar² is preferably:

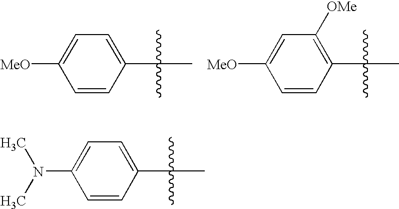

When unsubstituted, Ar² is preferably:

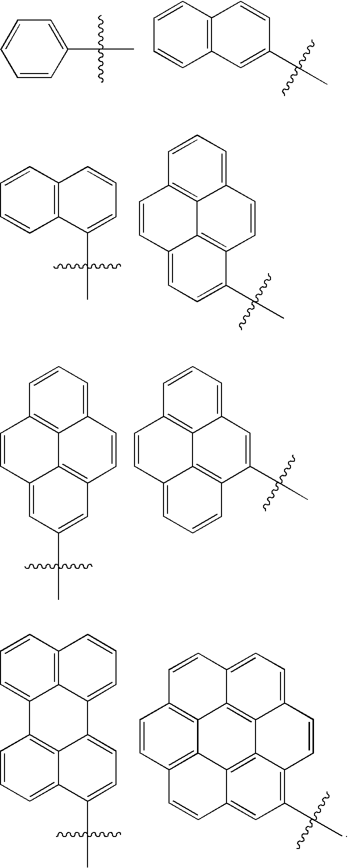

In another preferred embodiment, Ar² is cyclopropyl or cyclopropyl substituted with one or more B¹. Unsubstituted cyclopropyl is preferred. One or more, preferably one, of Ar² may be cyclopropyl.

X¹

Preferably group Xl is halogen, hydroxy, C₁₋₈hydrocarbyloxy, C₁₋₈hydrocarbyloxy substituted with one or more B¹, C₁₋₈heterohydrocarbyloxy, C₁₋₈heterohydrocarbyloxy substituted with one or more B¹, mesyl, tosyl, pentafluorophenyl, —O-succinimidyl —S-succinimidyl, or phenyloxy substituted with one or more B¹ e.g. p-nitrophenyloxy. The groups pentafluorophenyl, —O-succinimidyl, —S-succinimidyl, and p-nitrophenyloxy are preferred.

L

L is a linker group or a single covalent bond. Where L is a linker group it has a sufficient number of linking covalent bonds to link L to the nitrogen atom in formula (2) by a single covalent bond (or more, as appropriate) and to link L to the p instances of M groups (which may be attached to L by one or more bonds).

The group L is bonded to directly to the nitrogen atom in formula (2).

Preferred linker groups are -E^{M}-, -(D^{M})ₜ-, -(E^{M}-D^{M})ₜ-, -(D^{M}-E^{M})ₜ-, -E^{M}-(D^{M}-E^{M})ₜ- or -D^{M}-(E^{M}-D^{M})ₜ-, where a sufficient number of linking covalent bonds, in addition to the covalent bonds at the chain termini shown, are provided on groups E^{M} and D^{M} for linking the p instances of M groups.

D^{M} is independently C₁₋₈hydrocarbylene or C₁₋₈hydrocarbylene substituted with one or more B¹. Preferred D^{M} are C₁₋₈alkylene, C₁₋₈alkenylene and C₁₋₈alkynylene, especially C₁₋₈alkylene and C₁₋₈alkynylene, each optionally substituted with one or more A (preferably unsubstituted). A preferred substituent B¹ is ²H. Preferred L groups are: —CH₂CH₂—; —C≡C—CH₂CH₂CH₂—; —(CH₂)₅—; —CD₂CD₂CH₂CH₂CH₂—; —C≡C—CH₂— and —CH₂CH₂CH₂—.

E^{M} is independently -Z^{M}-, —C(=Z^{M})-, Z^{M}C(Z^{M})-, C(=Z^{M})Z^{M}-, Z^{M}C(=Z^{M})Z^{M}-, —S(═O)—, -Z^{M}S(═O)—, —S(═O)Z^{M}-, -Z^{M}S(═O)Z^{M}-, —S(═O)₂—, -Z^{M}S(═O)₂—, —S(═O)₂Z^{M}-, -Z^{M}S(═O)₂Z^{M}-, where Z^{M} is independently O, S or N(R^{M}) and where R^{M} is independently H, C₁₋₈hydrocarbyl (e.g C₁₋₈alkyl) or C₁₋₈hydrocarbyl substituted with one or more B¹. Preferably E^{M} is —O—, —S—, —C(═O)—, —C(═O)—, —C(═S)—, —C(═S)O—, —OC(═S)—, —C(═O)S—, —SC(═O)—, —S(O)—, —S(O)₂—, —NR^{M}—, C(═O)N(R^{M})—, —C(═S)N(R^{M})—, —N(R^{M})C(═O)—, —N(R^{M})C(═S)—, —S(═O)N(R^{M})—, —N(R^{M})S(═O)—, —S(═O)₂N(R^{M})—, —N(R^{M})S(═O)₂—, —OC(═O)O—, —SC(═O)O—, —OC(═O)S—, —N(R^{M})C(═O)O—, —OC(═O)N(R^{M})—, —N(R^{M})C(═O)N(R^{M})—, —N(R^{M})C(═S)N(R^{M})—, —N(R^{M})S(═O)N(R^{M})— or —N(R^{M})S(═O)₂N(R^{M})—.

Alternative groups E^{M} to those defined above, are -Z^{M}-Si(R^{M})₂-Z^{M}-, Si(R^{M})₂-Z^{M}- and -Z^{M}-Si(R^{M})₂—. The group —Si(R^{M})₂-Z^{M}- is particularly preferred. Z^{M} is preferably O. R^{M} is preferably C₁₋₈alkyl, preferably methyl. These groups E^{M} are particularly preferred in the groups -(E^{M}-D^{M})ₜ-, especially when t=1 and D^{M} is C₁₋₈alkylene. The following group is especially preferred:

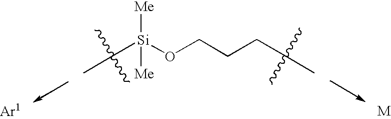

In addition to the above definition of D^{M}, D^{M} may also be C₁₋₈heterohydrocarbylene or C₁₋₈heterohydrocarbylene substituted with one or more B¹. In this embodiment, C₁₋₈cycloheteroalkylene groups are particularly preferred, e.g.:

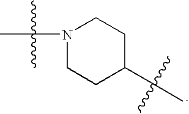

Thus, preferred L groups -D^{M}-E^{M}-D^{M}- are —C₁₋₈alkylene-C(O)—C₁₋₈cycloheteroalkylene (preferably where the hetero atom is N and is bound to the carboxy), especially:

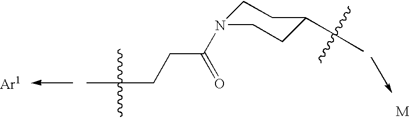

t=1 or more, e.g. from 1 to 50, 1 to 40, 1 to 30, 1 to 20 or 1 to 10. Preferably t=1, 2, 3, 4, 5, 6, 7, 8, 9, or 10.

Where L includes a group which also falls within the definition of group M, the group M is preferably more reactive than the group included in L

L is preferably -(D^{M})ₜ-, -(E^{M}-D^{M})ₜ-, or -D^{M}-(E^{M}-D^{M})ₜ-.

When group L is -(D^{M})ₜ-, t is preferably 1. D^{M} is preferably C₁₋₈alkylene, preferably C₁₋₅alkylene, preferably methylene or ethylene.

When group L is -(E^{M}-D^{M})ₜ-, or -D^{M}-(E^{M}-D^{M})ₜ-, E_{M} is preferably —C(═O)N(R^{M})- (e.g. —C(═O)NH—) or O (preferably O), and D^{M} is preferably C₁₋₈alkylene, preferably ethylene, propylene, butylene or pentylene. t is preferably 1. Especially preferred L are —O—CH₂CH₂CH₂— and —O—CH₂CH₂CH₂CH₂CH₂—.

Another preferred group -D^{M}-(E^{M}-D^{M})ₜ- is where D^{M} is C₁₋₈alkylene and t is 1. Preferred E^{M} in this group are -Z^{M}C(=Z^{M})- (especially —N(R^{M})C(═O)—, e.g. —N(Me)C(═O)—) and —C(=Z^{M})Z^{M}- (especially —C(═O)O—). Particularly preferred L groups are:

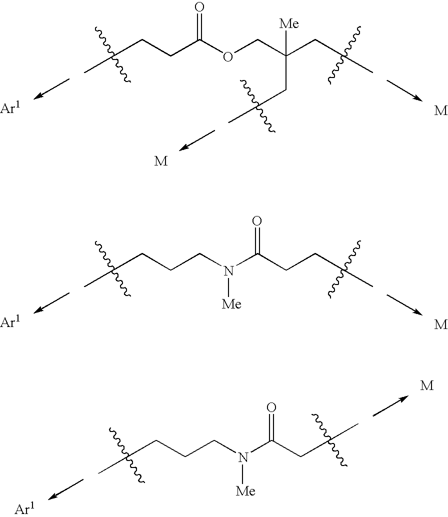

The group -(E^{M}-D^{M})ₜ- is preferred, a particularly preferred example of which —C(═O)NH—CH₂CH₂CH₂—O—CH₂CH₂—O—CH₂CH₂—O—CH₂CH₂CH₂—.

The group -(D^{M}-E^{M})ₜ- is also preferred when D^{M} is C₁₋₈alkylene and t is 1. Preferred E^{M} in this group, are -Z^{M}C(=Z^{M})- and —C(=Z^{M})Z^{M}-, especially -Z^{M}C(=Z^{M})- (particularly —N(R^{M})C(═O)—, e.g. —N(Me)C(═O)—). A particularly preferred example is —CH₂CH₂CH₂N(Me)C(O)—.

In an alternative embodiment it is preferred that L is a single covalent bond.

In a particularly preferred embodiment, L is methylene and M is a phosphoramidite group.

p

P is an integer having a value in the range from 1 to 10. Preferably p is 1, 2 or 3. Preferably p is 1.

Amino-Modified Biopolymers

As has been described above, the amino modifiers of the present invention are particularly advantageous for the synthesis of amino-modified biomolecules, in particular polynucleotides. It has surprisingly been found that when modified with the amino modifiers of the present invention, biomolecules have a long storage lifetime. In addition, advantageously, as the trityl protecting group on the amino modifier can be removed easily, it does not require stringent conditions in order to activate the amino-modified biomolecule for further reaction.

In addition, it has surprisingly been found that polynucleotides modified with the amino-modifiers of the present invention can be purified using the cheap and fast reverse phase purification technique and a high yield is obtained. This is a significant improvement as compared to amino modifiers which are currently commercially available. This is as a direct consequence of the balance between the stability of the trityl cation and the ease with which it can be removed from a biomolecule as defined in the present invention.

Amino-modified biomolecules can be formed by reacting the amino modifiers of the present invention having a structure (2) with a biomolecule having at least one group capable of reacting with M to form a covalent linkage to form a compound of formula (3).

The term ‘biomolecule’ includes polymers found in biological samples, including polypeptides, polysaccharides, and polynucleotides (e.g. DNA or RNA). Polypeptides may be simple copolymers of amino acids, or they may include post-translational modifications e.g. glycosylation, lipidation, phosphorylation, etc. Polynucleotides may be single-stranded (in whole or in part), double-stranded (in whole or in part), DNA/RNA hybrids, etc. RNA may be mRNA, rRNA or tRNA.

Preferably the biomolecule is a polynucleotide.

Polynucleotides

The polynucleotides used in the present invention may be of any suitable length. In particular, the polynucleotides may contain between 10 and 200 nucleotides.

Chemical Groups

The term ‘linker group’ includes any divalent group.

The term ‘halogen’ includes fluorine, chlorine, bromine and iodine.

The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₄ hydrocarbyl, more preferably C₁₋₈ hydrocarbyl.

Unless indicated explicitly otherwise, where combinations of groups are referred to herein as one moiety, e.g. arylalkyl, the last mentioned group contains the atom by which the moiety is attached to the rest of the molecule.

The term ‘hydrocarbylene’ includes linear, branched or cyclic divalent groups consisting of carbon and hydrogen formally made by the removal of two hydrogen atoms from the same or different (preferably different) skeletal atoms of the group. Hydrocarbylene groups thus include alkylene, alkenylene and alkynylene groups, cycloalkylene (including polycycloalkylene), cycloalkenylene and arylene groups and combinations thereof, e.g. alkylenecycloalkylene, alkylenepolycycloalkylene, alkylenearylene, alkenylenearylene, cycloalkylenealkylene, polycycloalkylenealkylene, arylenealkylene and arylenealkenylene groups. Preferred hydrocarbylene are C₁₋₁₄ hydrocarbylene, more preferably C₁₋₈ hydrocarbylene.

The term ‘hydrocarbyloxy’ means hydrocarbyl-O—.

The terms ‘alkyl’, ‘alkylene’, ‘alkenyl’, ‘alkenylene’, ‘alkynyl’, or ‘alkynylene’ are used herein to refer to both straight, cyclic and branched chain forms. Cyclic groups include C₃₋₈ groups, preferably C₅₋₈ groups.

The term ‘alkyl’ includes monovalent saturated hydrocarbyl groups. Preferred alkyl are C₁₋₁₀, more preferably C₁₋₄ alkyl such as methyl, ethyl, n-propyl, i-propyl or t-butyl groups.

Preferred cycloalkyl are C₅₋₈ cycloalkyl.

The term ‘alkoxy’ means alkyl-O—.

The term ‘alkenyl’ includes monovalent hydrocarbyl groups having at least one carbon-carbon double bond and preferably no carbon-carbon triple bonds. Preferred alkenyl are C₂₋₄ alkenyl.

The term ‘alkynyl’ includes monovalent hydrocarbyl groups having at least one carbon-carbon triple bond and preferably no carbon-carbon double bonds. Preferred alkynyl are C₂₋₄ alkynyl.

The term ‘aryl’ includes monovalent aromatic groups, such as phenyl or naphthyl. In general, the aryl groups may be monocyclic or polycyclic fused ring aromatic groups. Preferred aryl are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘alkylene’ includes divalent saturated hydrocarbylene groups. Preferred alkylene are C₁₋₄ alkylene such as methylene, ethylene, n-propylene, i-propylene or t-butylene groups.

Preferred cycloalkylene are C₅₋₈ cycloalkylene.

The term ‘alkenylene’ includes divalent hydrocarbylene groups having at least one carbon-carbon double bond and preferably no carbon-carbon triple bonds. Preferred alkenylene are C₂₋₄ alkenylene.

The term ‘alkynylene’ includes divalent hydrocarbylene groups having at least one carbon-carbon triple bond and preferably no carbon-carbon double bonds. Preferred alkynylene are C₂₋₄ alkynylene.

The term ‘arylene’ includes divalent aromatic groups, such phenylene or naphthylene. In general, the arylene groups may be monocyclic or polycyclic fused ring aromatic groups. Preferred arylene are C₆-C₁₄arylene.

Other examples of arylene groups are divalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘heterohydrocarbyl’ includes hydrocarbyl groups in which up to three carbon atoms, preferably up to two carbon atoms, more preferably one carbon atom, are each replaced independently by O, S, Se or N, preferably O, S or N. Heterohydrocarbyl groups thus include heteroalkyl, heteroalkenyl and heteroalkynyl groups, cycloheteroalkyl (including polycycloheteroalkyl), cycloheteroalkenyl and heteroaryl groups and combinations thereof, e.g. heteroalkylcycloalkyl, alkylcycloheteroalkyl, heteroalkylpolycycloalkyl, alkylpolycycloheteroalkyl, heteroalkylaryl, alkylheteroaryl, heteroalkenylaryl, alkenylheteroaryl, cycloheteroalkylaryl, cycloalkylheteroaryl, heterocycloalkenylaryl, cycloalkenylheteroaryl, cycloalkylheteroalkyl, cycloheteroalkylalkyl, polycycloalkylheteroalkyl, polycycloheteroalkylalkyl, arylheteroalkyl, heteroarylalkyl, arylheteroalkenyl, heteroarylalkenyl, arylcycloheteroalkyl, heteroarylcycloalkyl, arylheterocycloalkenyl and heteroarylcycloalkenyl groups. The heterohydrocarbyl groups may be attached to the remainder of the compound by any carbon or hetero (e.g. nitrogen) atom.

The term ‘heterohydrocarbylene’ includes hydrocarbylene groups in which up to three carbon atoms, preferably up to two carbon atoms, more preferably one carbon atom, are each replaced independently by O, S, Se or N, preferably O, S or N. Heterohydrocarbylene groups thus include heteroalkylene, heteroalkenylene and heteroalkynylene groups, cycloheteroalkylene (including polycycloheteroalkylene), cycloheteroalkenylene and heteroarylene groups and combinations thereof, e.g. heteroalkylenecycloalkylene, alkylenecycloheteroalkylene, heteroalkylenepolycycloalkylene, alkylenepolycycloheteroalkylene, heteroalkylenearylene, alkyleneheteroarylene, heteroalkenylenearylene, alkenyleneheteroarylene, cycloalkyleneheteroalkylene, cycloheteroalkylenealkylene, polycycloalkyleneheteroalkylene, polycycloheteroalkylenealkylene, aryleneheteroalkylene, heteroarylenealkylene, aryleneheteroalkenylene, heteroarylenealkenylene groups. The heterohydrocarbylene groups may be attached to the remainder of the compound by any carbon or hetero (e.g. nitrogen) atom.

Where reference is made to a carbon atom of a hydrocarbyl or other group being replaced by an O, S, Se or N atom, what is intended is that:

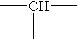

is replaced by

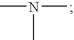

—CH═ is replaced by —N═; or

—CH₂— is replaced by , —S— or —Se—.

The term ‘heteroalkyl’ includes alkyl groups in which up to three carbon atoms, preferably up to two carbon atoms, more preferably one carbon atom, are each replaced independently by O, S, Se or N, preferably O, S or N.

The term ‘heteroalkenyl’ includes alkenyl groups in which up to three carbon atoms, preferably up to two carbon atoms, more preferably one carbon atom, are each replaced independently by O, S, Se or N, preferably O, S or N.

The term ‘heteroalkynyl’ includes alkynyl groups in which up to three carbon atoms, preferably up to two carbon atoms, more preferably one carbon atom, are each replaced independently by O, S, Se or N, preferably O, S or N.

The term ‘heteroaryl’ includes aryl groups in which up to three carbon atoms, preferably up to two carbon atoms, more preferably one carbon atom, are each replaced independently by O, S, Se or N, preferably O, S or N. Preferred heteroaryl are C₅₋₁₄heteroaryl. Examples of heteroaryl are pyridyl, pyrrolyl, thienyl or furyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The term ‘heteroalkylene’ includes alkylene groups in which up to three carbon atoms, preferably up to two carbon atoms, more preferably one carbon atom, are each replaced independently by O, S, Se or N, preferably O, S or N.

The term ‘heteroalkenylene’ includes alkenylene groups in which up to three carbon atoms, preferably up to two carbon atoms, more preferably one carbon atom, are each replaced independently by O, S, Se or N, preferably O, S or N.

The term ‘heteroalkynylene’ include alkynylene groups in which up to three carbon atoms, preferably up to two carbon atoms, more preferably one carbon atom, are each replaced independently by O, S, Se or N, preferably O, S or N.

The term ‘heteroarylene’ includes arylene groups in which up to three carbon atoms, preferably up to two carbon atoms, more preferably one carbon atom, are each replaced independently by O, S, Se or N, preferably O, S or N. Preferred heteroarylene are C₅₋₁₄heteroarylene. Examples of heteroarylene are pyridylene, pyrrolylene, thienylene or furylene.

Other examples of heteroarylene groups are divalent derivatives (where the valency is adapted to accommodate the q instances of the linker L) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroarylene groups are five- and six-membered divalent derivatives, such as the divalent derivatives of furan, imidazole, isothiazole, isoxazole, pyran, pyrazine, pyrazole, pyridazine, pyridine, pyrimidine, pyrrole, pyrrolizine and thiophene. The five-membered divalent derivatives are particularly preferred, i.e. the divalent derivatives of furan, imidazole, isothiazole, isoxazole, pyrazole, pyrrole and thiophene.

Substitution

B¹ is independently a substituent, preferably a substituent S_{ub}¹. Alternatively, B¹ may be ²H.

S_{ub}¹ is independently halogen, trihalomethyl, —NO₂, —CN, —N⁺(R⁵)₂O⁻, —CO₂H, —CO₂R⁵, —SO₃H, —SOR⁵, —SO₂R⁵, —SO₃R⁵, —OC(═O)OR⁵, —C(═O)H, —C(═O)R⁵, —OC(═O)R⁵, —NR⁵₂, —C(═O)NH₂, —C(═O)NR⁵₂, —N(R⁵)C(═O)OR⁵, —N(R⁵)C(═O)NR⁵₂, —OC(═O)NR⁵₂, —N(R⁵)C(═O)R⁵, —C(═S)NR⁵₂, —NR⁵C(═S)R⁵, —SO₂NR⁵₂, —NR⁵SO₂R⁵, —N(R⁵)C(═S)NR⁵₂, —N(R⁵)SO₂NR⁵₂, —R⁵ or -Z²R⁵.

Z² is O, S, Se or NR⁵.

R⁵ is independently H, C₁₋₈hydrocarbyl, C₁₋₈hydrocarbyl substituted with one or more S_{ub}², C₁₋₈heterohydrocarbyl or C₁₋₈heterohydrocarbyl substituted with one or more S_{ub}².

S_{ub}² is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z²C₁₋₆alkyl.

Where reference is made to a substituted group, the substituents are preferably from 1 to 5 in number, most preferably 1.

General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

Figures

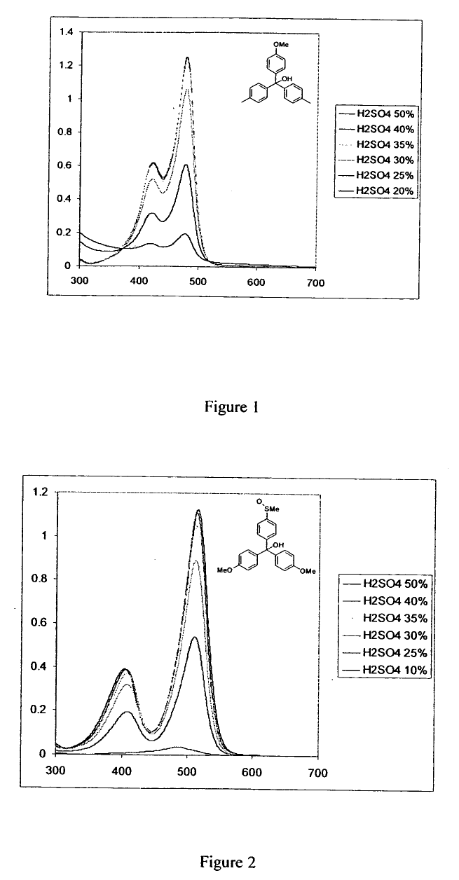

FIG. 1 is a graph showing absorbance against wavelength for MDMeTrOH; and

FIG. 2 is a graph showing absorbance against wavelength for DMS(O)TrOH

### EXAMPLES

### Example 1

### (4-methoxy-4′,4″-dimethyltrityl alcohol (MDMeTrOH))

4,4′-dimethylbenzophenone (2.10 g, 10 mmol) was dissolved in dry THF (100 mL) and a 0.5 M 4-methoxyphenylmagnesium bromide solution in THF (30 mL, 15 mmol) was added under N₂ within 5 min. The mixture was left overnight ambient temperature. TLC (1:1 CH₂Cl₂/hexane) showed complete conversion of starting ketone. The mixture was quenched with 10 mL of asaturated NH₄Cl solution, evaporated, partitioned between brine (150 mL) and EtOAc (150 mL); and the organic layer was washed with brine (100 mL), dried over Na₂SO₄, and evaporated. The residue was purified with a silica gel column (15×4.5 cm). The desired compound was eluted with 0 to 1.5% EtOAc in toluene. Fractions containing product were evaporated, and the residue was filtered, washed with hexane, and dried in vacuo. Yield 2.86 g (90%). The synthesis is shown schematically in scheme 1 below:

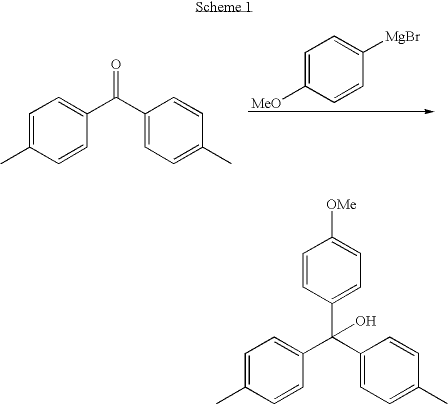

Solutions of MDMeTrOH were prepared in acetic acid such that each solution had a concentration in the range from 10⁻⁵ to 10⁻⁷, A constant amount of the acetic acid solution prepared in this way was then added to the solutions of sulphuric acid by use of, for example, a micropipette. Using UV spectrometry, the λₘₐₓ of the trityl cation was determined to be approximately 477 nm, as illustrated in FIG. 1. Absorbance values were recorded at this wavelength for different concentrations of sulphuric acid. The results are shown in table 1 below.

TABLE 1

| | H₂SO₄ 50% | H₂SO₄ 40% | H₂SO₄ 35% | H₂SO₄ 30% | H₂SO₄ 25% | H₂SO₄ 20% |
|---|---|---|---|---|---|---|
| Max Abs | 1.252 | 1.25 | 1.227 | 1.061 | 0.608 | 0.195 |
| at (nm) | 477.5 | 477.5 | 477 | 477 | 477 | 475.5 |
| | | | pK_{R+} (477 nm) | | | |
| | | H₂SO₄ 30% | −2.5 | | | |
| | | H₂SO₄ 25% | −2.6 | | | |
| | | H₂SO₄ 20% | −2.7 | | | |

pK_{R+} = −2.6

As shown in Table 1, MDMeTrOH was found to have a pK_{R+} of approximately −2.6.

An amino modifier was then synthesised using MDMeTrOH as the starting reagent as shown schematically in scheme 2 below.

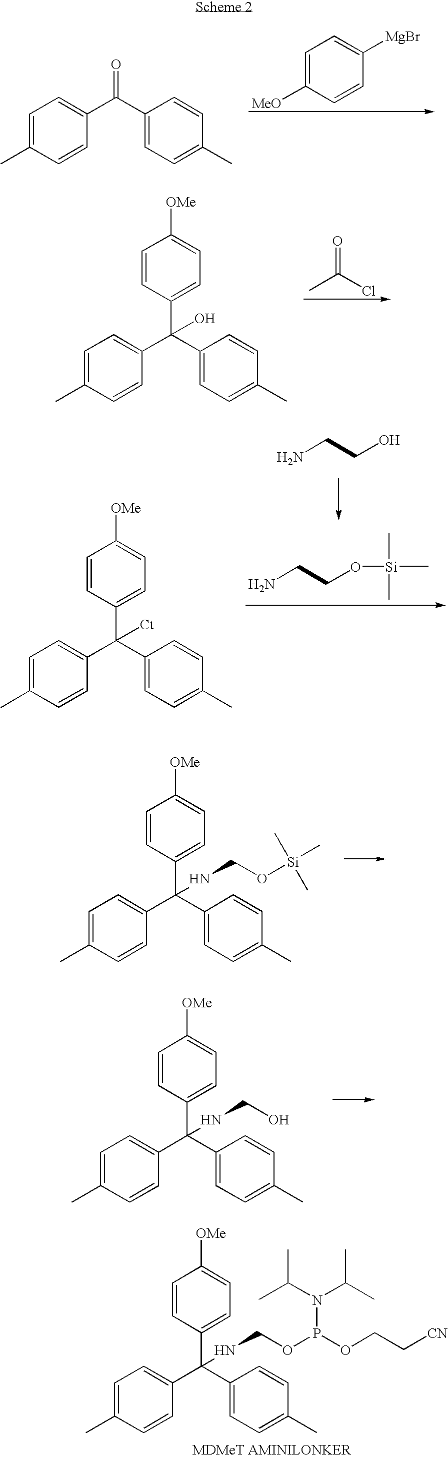

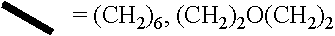

The experimental details for each stage of this synthesis are as follows.

### 4-methoxy-4′,4″-dimethyltrityl chloride

4-methoxy-4′,4″-dimethyltrityl alcohol (3.18 g, 10 mmol) was dissolved in a 50 mL 50% solution of freshly distilled acetyl chloride in dry toluene. The solution was refluxed for 1 h and then it was allowed to cool down to room temperature. Volatiles were then evaporated under reduced pressure (10 mmHg) and the residue was twice azeotroped with dry toluene. The obtained 4-methoxy-4′,4″-dimethyltrityl chloride (3.36 g, 10 mmol) was used without any further purification (Yield 100%). The reaction is shown schematically in scheme 3 below.

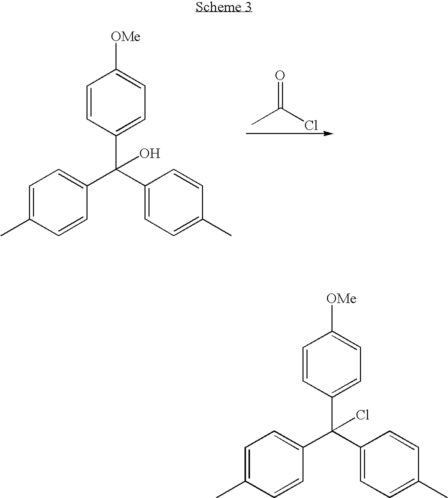

### 6-(4-methoxy-4′,4″-dimethyltritylamino)hexanol

A solution of 6-aminohexanol (5.38 g, 4.6 mmol) and triethylamine (1.60 mL, 11.5 mmol) was treated with trimethylchlorosilane (0.6 mL, 46 mmol). The mixture was stirred for 10 min and a solution of 4-methoxy-4′,4″-dimethyltrityl choride (4.6 mmol, 1.54 g) dichloromethane was then dropwise added.

The resulting solution was kept overnight, and tetrabutylammonium fluoride trihydrate (1.59 g, 5.1 mmol) was added. The mixture was then evaporated, dissolved in EtOAc (50 mL), washed with saturated aqueous NaHCO₃ (30 mL), water (30 mL), brine (30 mL), dried over anhydrous Na₂SO₄ and evaporated. The residue was chromatographed on silica gel (10% to 40% acetone and 1% triethylamine in toluene) to provide the title compound in a yield of 1.54 g (80%). The reaction is shown schematically in scheme 4 below.

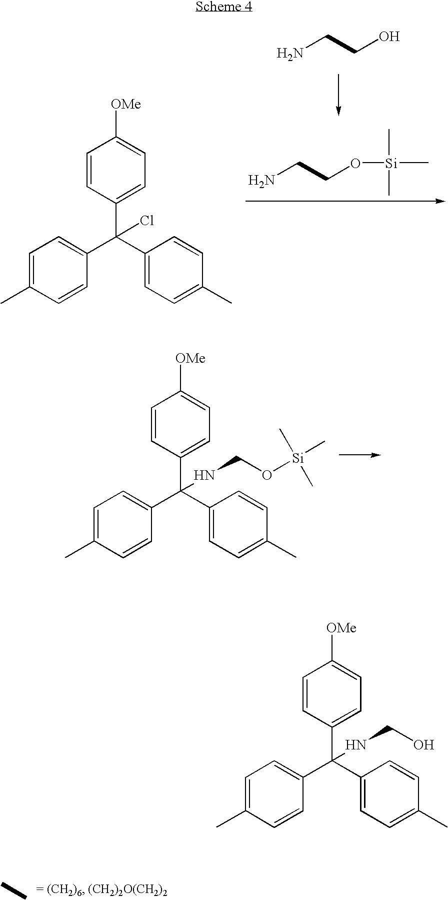

### 6-(4-methoxy-4′,4″-dimethyltritylamino)hexylolxy-(2-cyanoethoxy) diisopropylaminophosphine

Bis(diisopropylamino)-(2-cyanoethoxy)phosphine (1.36 g, 4.5 mmol) was added under argon to a solution of 6-(4-methoxy-4′,4″-dimethyltrityl amino)hexanol (1.56 g, 3.75 mmol) and diisopropylammonium tetrazolide (0.96 g, 5.6 mmol) in dichloromethane (20 mL). The mixture was stirred at room temperature under argon overnight and diluted with dichloromethane (40 mL), washed with saturated aqueous NaHCO₃ (30 mL) and water (30 mL), dried over Na₂SO₄ and evaporated. The residue was subjected to column chromatography on silica gel (20% acetone and 2% triethylamine in toluene) to give the amino modifier compound in a yield of 2.5 g (90%). The reaction is shown schematically in scheme 5 below.

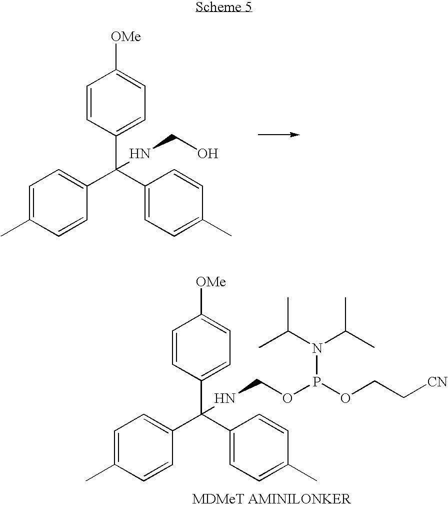

### Example 2

### (4-methylsulfinyl-4′,4″-dimethoxytrityl alcohol (DMS(O)MTrOH))

4,4′-dimethoxybenzophenone (2.42 g, 10 mmol) was dissolved in dry THF (100 mL) and a 0.5 M 4-Thioanisolemagnesium bromide solution in THF (30 mL, 15 mmol) was added under N₂ within 5 min. The mixture was left overnight ambient temperature. TLC (1:1 CH₂Cl₂/hexane) showed complete conversion of starting ketone. The mixture was quenched with 10 mL of a saturated NH₄Cl solution, evaporated, partitioned between brine (150 mL) and EtOAc (150 mL); and the organic layer was washed with brine (100 mL), dried over Na₂SO₄, and evaporated. The residue was purified with a silica gel column (15×4.5 cm). The desired compound was eluted with 0→1.5% EtOAc in toluene. Fractions containing product were evaporated, and the residue was filtered, washed with hexane, and dried in vacuo. Yield 2.92 g (80%). The reaction is shown schematically in scheme 6 below.

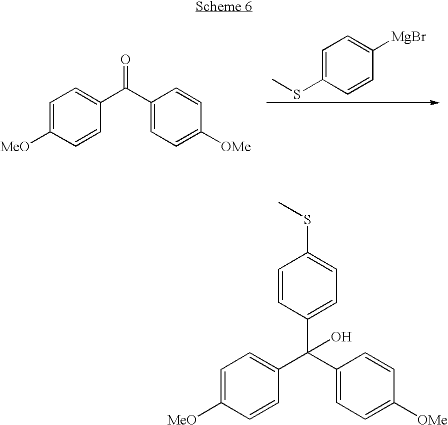

Solutions of DMS(O)MTrOH were prepared in acetic acid such that each of the solutions has a concentration in the range from 10⁻⁵ to 10⁻⁷, A constant amount of the acetic acid solution prepared in this way was then added to the solutions of sulphuric acid by use of, for example, a micropipette. Using UV spectrometry, the λₘₐₓ of the trityl cation was determined to be approximately 512 nm, as can be seen from FIG. 2. Absorbance values were then recorded at this wavelength for different concentrations of sulphuric acid. The results are shown in table 2 below.

TABLE 2

| | H₂SO₄ 50% | H₂SO₄ 40% | H₂SO₄ 35% | H₂SO₄ 30% | H₂SO₄ 25% | H₂SO₄ 10% |
|---|---|---|---|---|---|---|
| max abs | 1.124 | 1.109 | 1.048 | 0.891 | 0.541 | 0.128 |
| at nm | 514 | 512.5 | 512 | 511 | 510 | 252 |
| | | pKR+ (512 nm) | | | | |
| | H₂SO₄ 30% | −2.6 | | | | |
| | H₂SO₄ 25% | −2.6 | | | | |

pKR+ = −2.6

An amino modifier was then synthesised using DMS(O)MTrOH as the starting reagent as shown schematically in scheme 7 below.

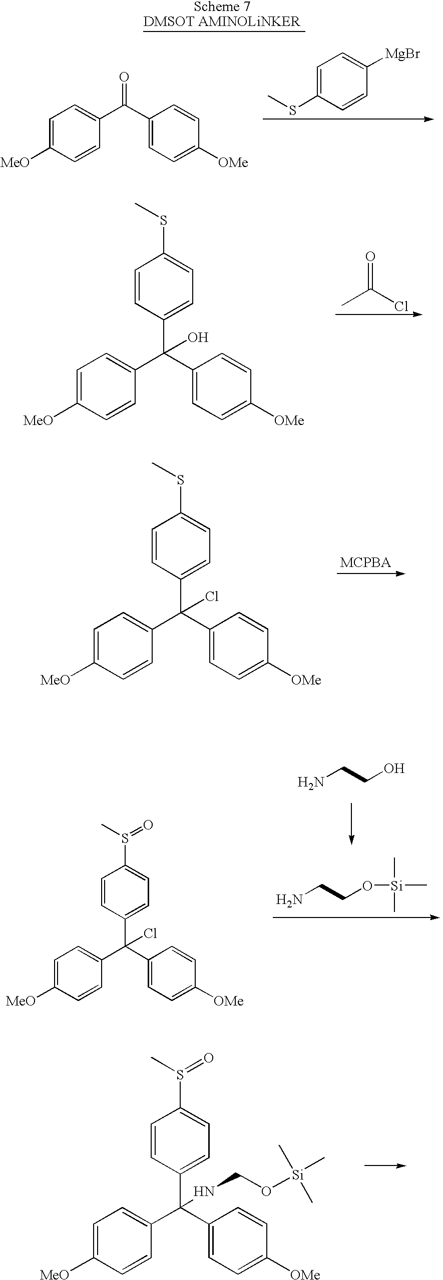

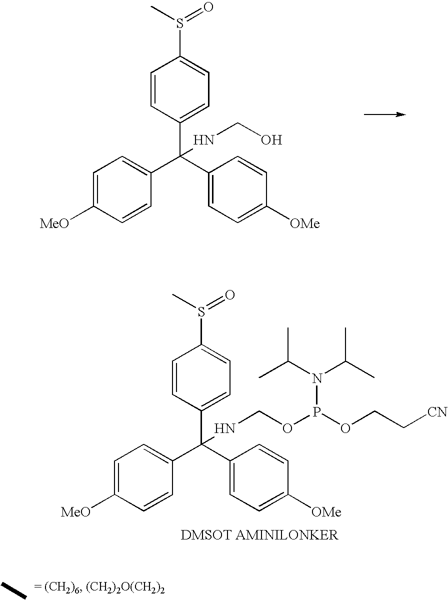

The experimental details for each stage of this synthesis are as follows.

### 4,4′-dimethoxy-4″-(methylthio)trityl chloride

4,4′-dimethoxy-4″-(methylthio)trityl alcohol (36.6 g, 100 mmol) was dissolved in a 300 mL 50% solution of freshly distilled acetyl chloride in dry toluene. The solution was refluxed for I h and then it was allowed to cool down to room temperature. Volatiles were then evaporated under reduced pressure (10 mmHg) and the residue was twice azeotroped with dry toluene. The obtained 4,4′-dimethoxy-4″-(methylthio)trityl chloride (38.0 g, 10 mmol) was used without any further purification (Yield 100%). The reaction is shown schematically in scheme 8 below.

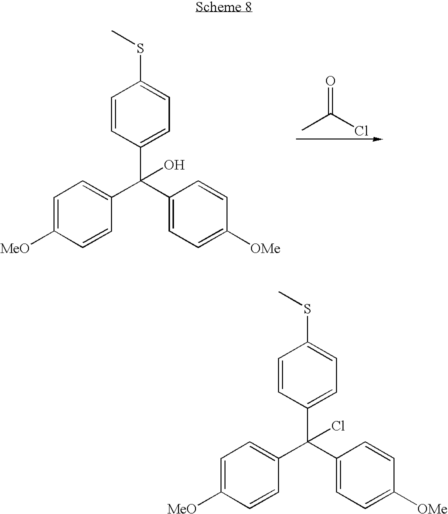

### 4,4′-dimethoxy-4″-(methylsulfinyl)trityl chloride

A suspension of 3-chloroperbenzoic acid (8.71 g, 50.5 mmol) in dichloromethane (100 mL) was added dropwise to a stirred solution of 4,4′-dimethoxy-4″-(methylthio)trityl chloride (17.66 g, 45.9 mmol) in dichloromethane (50 mL). After 15 min at rt, the mixture was cooled to −30° C., and filtered under argon to yield a clear solution of 4,4′-dimethoxy-4″-(methylsulfinyl)trityl chloride. The reaction is shown schematically in scheme 9 below.

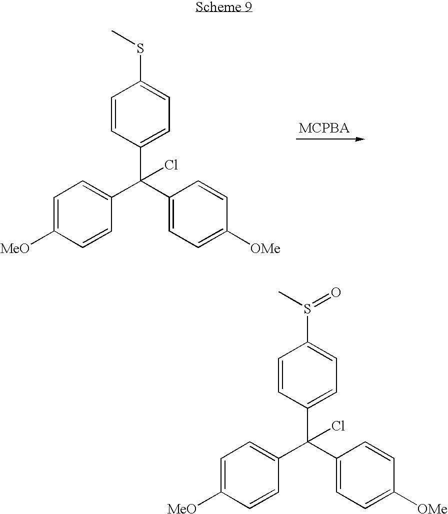

### 6-(4,4′-dimethoxy-4″-(methylsulfinyl)tritylamino)hexanol

A solution of 6-aminohexanol (5.38 g, 45.9 mmol) and triethylamine (16.0 mL, 115 mmol) was treated with trimethylchlorosilane (5.82 mL, 45.9 mmol). The mixture was stirred for 10 min and the previously prepared (see avobe) solution of 4,4′-dimethoxy-4″-(methylsulfinyl)trityl chloride was dropwise added.

The resulting solution was kept overnight, and tetrabutylammonium fluoride trihydrate (15.90 g, 51 mmol) was added. The mixture was then evaporated, dissolved in EtOAc (500 mL), washed with saturated aqueous NaHCO₃ (300 mL), water (300 mL), brine (300 mL), dried over Na₂SO₄ and evaporated. The residue was chromatographed on silica gel (10% to 40% acetone and 1% triethylamine in toluene) to provide the title compound. Yield 18.07 g (82%). The reaction is shown schematically in scheme 10 below.

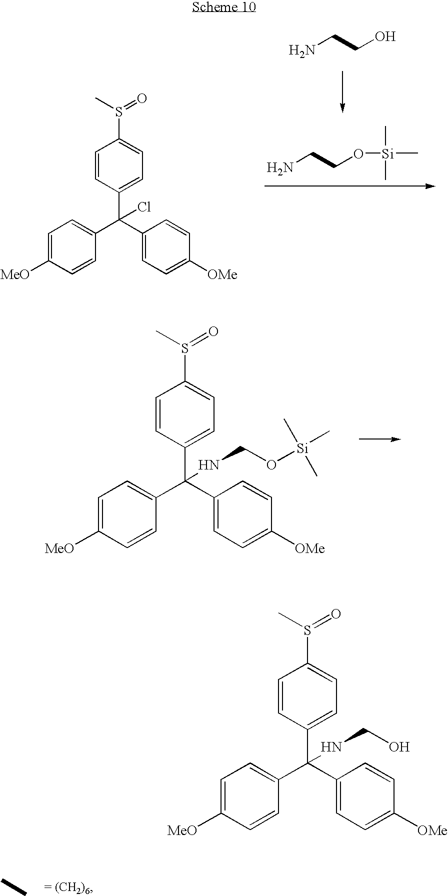

### 6-(4,4′-dimethoxy-4″-(methylsulfinyl)tritylamino)hexylolxy-(2-cyanoethoxy) diisopropylaminophosphine

Bis(diisopropylamino)-(2-cyanoethoxy)phosphine (13.57 g, 45.0 mmol) was added under argon to a solution of 6-(4,4′-dimethoxy-4″-(methylsulfinyl)tritylamino)hexanol (18.07 g, 37.5 mmol) and diisopropylammonium tetrazolide (9.64 g, 56.3 mmol) in dichloromethane (200 mL). The mixture was stirred at room temperature under argon overnight and diluted with dichloromethane (400 mL), washed with saturated aqueous NaHCO₃ (300 mL) and water (300 mL), dried over Na₂SO₄ and evaporated. The residue was subjected to column chromatography on silica gel (20% acetone and 2% triethylamine in toluene) to give the title compound. Yield 23.18 g (91%). The reaction is shown schematically in scheme 11 below.

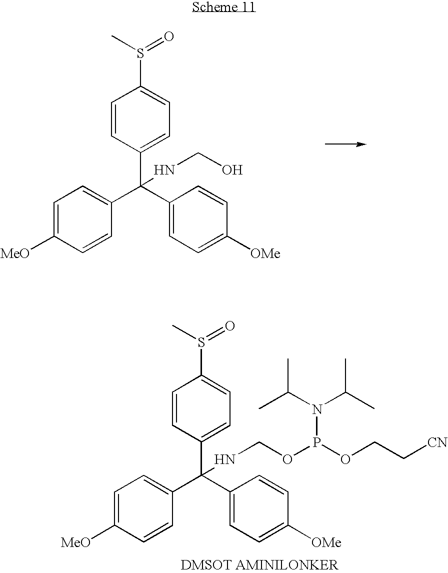

### Evaluation of the Amino-Modifiers in the Polynucleotide Synthesis Context

A real world sequence for the T7 Universal primer was used. This is a 19 mer mixed base sequence. The DMS(O)MTr amino modifier was coupled to the 5′ end of four 1-μmole syntheses and cleaved/deprotected with NH₄OH. It was filtered and the two syntheses were combined from 2 μmole and diluted to 5 ml with NH₄OH and then further diluted with 15 ml H₂O to a final volume of 20 ml.

Experiments were then performed using PolyPak available from Glen Research (Virginia, USA) barrels and 2-2.5 ml of polynucleotide sample (0.2 to 0.25 μmole equivalents).

### Experiment 1

The first series of experiments used the standard protocol, as provided with the PolyPak product, for PolyPak purification except that a 4% solution of TFA for 5 minutes was used and the purified polynucleotide was eluted with 50% acetonitrile in 0.1 M TEAA to elute both trityl-on and trityl-off polynucleotide at neutral pH to retain any trityl that might still be on the polynucleotide. Three samples had the failures eluted with 1:20 NH₄OH and a second set of three used 10% ACN/0.1 M TEAA to elute the failures. The fractions containing unbound polynucleotide, 1:20 NH₄OH wash or 10% ACN/0.1 M TEAA wash and purified polynucleotide were analyzed by RP HPLC and recoveries of “Trityl-On” polynucleotides calculated based on peak area and volume.

### Results

1. PolyPac purification worked well with near total binding of Trityl-on polynucleotide in the loading step.

2. 1:20 NH₄OH wash was better at removing only failure sequences than 10% ACN wash. NH₄OH (1:20) wash eluted an average of only 1.4% trityl-on polynucleotide vs. 6.8% for the 10% ACN wash.

3. Final wash recovered 70-74% of the trityl-on polynucleotide, with the trityl removed, when the 1:20 NH₄OH wash was used (61-71% for the 10% ACN wash samples) and there was little if any trityl-on polynucleotide remaining.

### Experiment 2

The first experiment was repeated this time only using the 1:20 NH₄OH wash and comparing 2% TFA solution to 4% TFA solution for 5 minutes.

Samples were eluted either with 20% ACN in 0.1 M TEAA or 50% ACN in 0.1 M TEAA to compare recoveries.

### Results

1. Samples detritylated with the 2% TFA solution gave lower yields and had a significant 30% as trityl-on polynucleotide.

2. Results with 4% TFA were confirmed.

### Experiment 3

In this experiment, the standard protocol using 4% TFA for 5 minutes to detritylate the polynucleotide, either 1:20 NH₄OH or 10% ACN in 0.1 M TEAA for the wash and 20% ACN in H₂O to elute the polynucleotide was used.

### Results

1. This experiment confirmed all of the earlier results.

2. Polynucleotide bound nearly 100% in loading 3. NH₄OH (1:20) wash eluted less trityl-on polynucleotide (5.6% vs. 11%) 4. Final polynucleotide was eluted as trityl-off with a recovery of 77% for NH₄OH wash and 64% for 10% ACN wash.

### Experiment 4

To evaluate the stability of the DMS(O)MT-amine, the HPLC of the polynucleotide in NH₄OH one day after cleavage/deprotection was compared to the same sample stored at RT for 6 days. There was no difference in the per-cent trityl-on polynucleotide indicating that the trityl was stable under these conditions.

### Summary

The 4,4′-Dimethoxy-4″-thiomethoxytrityl (DMS(O)MTr) cation is more stabilized than the commercially available MMTr cation, and so the DMS(O)MTr-protected amino group is easier to deprotect than the the MMTr-protected one.

The sulfoxy derivative survives conditions of polynucleotide synthesis and can either be cleaved with standard deblock solution, or left intact for an HPLC purification. At the same time, the DMS(O)MTr group is fully compatible with cartridge purification: when detritylation on cartridge is carried out, the DMS(O)MTr+, which is more stable than MMTr+, does not reattach itself to an amine. The reagent is stable in acetonitrile at room temperature for at least two weeks. UV quantification for release of the new protecting group is possible. Extinction coefficients (L/(mol×cm) shown in the Scheme above were measured in 2% TFA/DCM.

In the PolyPak detritylation experiments followed by HPLC measurements, the new reagent gave more than 20% improvement in deprotection yields compared to an MMTr-protected amino group labeled polynucleotide (4% TFA, 5 min exposure time).

1. A compound of formula (1): 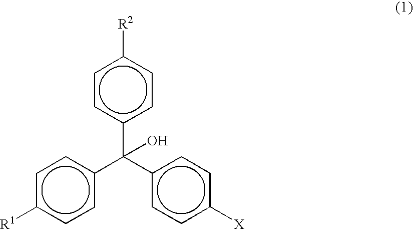
wherein: X is an electron-donating group;
R¹ and R² are each independently selected from hydrogen, halogen, C₁₋₁₀ hydrocarbyl, C₁₋₁₀ hydrocarbyl substituted with one or more A¹, C₂₋₁₀ hydrocarbylene, C₁₋₁₀ hydrocarbylene substitutued with one or more A¹, trihalomethyl, —NO₂, —CN, —N^{+(R}³)₂O⁻, —CO₂H, —CO₂R³, —SO₃H, —SOR³, —SO₂R³, —SO₃R³, —OC(═O)OR³, —C(═O)H, —C(═O)R³, —OC(═O)R³, —NR³₂, —C(═O)NH₂, —C(═O)NR³₂, —N(R³)C(═O)OR³, —N(R³)C(═O)NR³₂, —OC(═O)NR³₂, —N(R³)C(═O)R³, —C(═S)NR³₂, —NR³C(═S)R³, —SO₂NR³₂, —NR³SO₂R³, —N(R³)C(═S)NR³₂, —N(R³)SO₂NR³₂, —R³ or -Z¹R³;
Z¹ is O, S, Se or NR³;
R³ is independently H, C₁₋₁₀hydrocarbyl, C₁₋₁₀hydrocarbyl substituted with one or more A¹, C₁₋₁₀heterohydrocarbyl; C₁₋₁₀heterohydrocarbyl substituted with one or more A¹; C₂₋₁₀ hydrocarbylene; or C₂₋₁₀ hydrocarbylene substituted with one or more A¹;
A¹ is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z¹C₁₋₆alkyl; and
the compound has a pK_{R+} in the range from −3.10 to −1.50.

2. A compound according to claim 1, wherein X is —OC₁₋₆alkyl.

3. A compound according to claim 2, wherein X is —OCH₃.

4. A compound according to claim 3, wherein R¹ and R² are the same.

5. A compound according to claim 4, wherein R¹ and R² are methyl.

6. A compound according to claim 3, wherein R¹ and R² are different.

7. A compound according to claim 6, wherein R¹ is —OC₁₋₆alkyl and R² is —SOR³.

8. A compound according to claim 7, wherein R¹ is —OCH₃ and R² is —SOCH₃.

9. A compound according to claim 1, which has a pK_{R+} value in the range from −2.8 to −2.0.

10. A method of producing a compound of formula (1) comprising reacting a compound of formula (4): 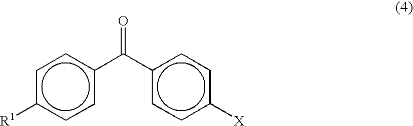
with a Grignard reagent of formula (5): 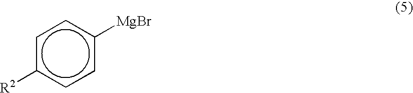
wherein X, R¹ and R² are as defined in claim 1.

11. An amino modifier of formula (2): 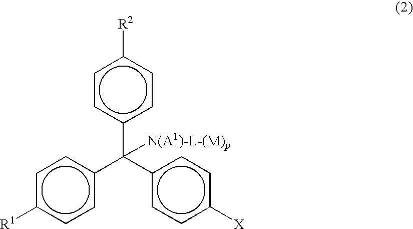
wherein
X, R¹ and R² are as defined in claim 1;
L is a linker group;
M is a reactive functional group which is capable of reacting with a biomolecule to form a covalent bond; and
p is an integer having a value in the range from 1 to 10.

12. An amino modifier according to claim 11, wherein X is —OC₁₋₆alkyl.

13. An amino modifier according to claim 12, wherein X is —OCH₃.

14. An amino modifier according to claim 13, wherein R¹ and R² are the same.

15. An amino modifier according to claim 14, wherein R¹ and R² are methyl.

16. An amino modifier according to claim 12, wherein R¹ and R² are different.

17. An amino modifier according to claim 14, wherein R¹ is —OC₁₋₆alkyl and R² is —SOR³.

18. An amino modifier according to claim 17, wherein R¹ is —OCH₃ and R² is —SOCH₃.

19. An amino modifier according to claim 11, wherein L is a C₁₋₁₀ alkyl group.

20. An amino modifier according to claim 19, wherein L is methylene.

21. An amino modifier according to claim 11 wherein M is a phosphoramidite group.

22. An amino modifier according to claim 21, wherein M is:

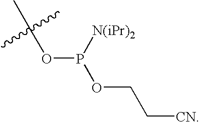

23. A method of producing an amino modifier of formula (2) comprising:
(a) reacting a compound of formula (1) with acetyl chloride;
(b) reacting the product of step (a) with an amino alcohol, wherein the hydroxyl group of the amino alcohol has been protected; and
(c) removing the protecting group from the hydroxyl group of the product of step (b) to produce a compound of formula (2).

24. A method of synthesising an amino-modified biomolecule comprising reacting an amino modifier as defined in claim 11 with a biomolecule.

25. An amino-modified biomolecule of formula (3): 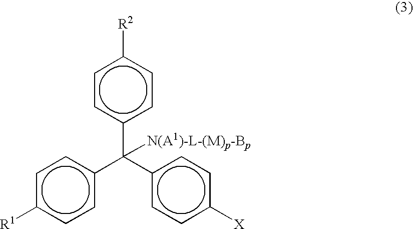
wherein X, R¹ and R², L and M are as defined in claim 11; and
Bₚ is a biomolecule.

26. An amino-modified biomolecule according to claim 24, wherein the biomolecule is an polynucleotide.

27. A method of producing an amino-modified biomolecule comprising reacting a compound of formula (2) with a biomolecule, Bₚ having at least one group capable of reacting with M to form a covalent linkage.
